(19) United States
(12) Patent Application Publication
(10) Pub. No.: US 2025/0078954 A1
(43) Pub. Date: Mar. 6, 2025

(54) JOINT PREDICTION OF ODORANT-OLFACTORY RECEPTOR BINDING AND ODORANT PERCEPTS

(71) Applicant: Microsoft Technology Licensing, LLC, Redmond, WA (US)

(72) Inventors: Judith AMORES FERNANDEZ, Somerville, MA (US); Seyone CHITHRANANDA, Berkeley, CA (US); Kevin Kaichuang YANG, Cambridge, MA (US)

(73) Assignee: Microsoft Technology Licensing, LLC, Redmond, WA (US)

(21) Appl. No.: 18/375,219

(22) Filed: Sep. 29, 2023

Related U.S. Application Data
(60) Provisional application No. 63/536,281, filed on Sep. 1, 2023.

(51) Int. Cl. G16B 15/30

(52) U.S. Cl. CPC G16B 15/30

(57) ABSTRACT

Systems and methods for determining predicted olfactory perception are provided. In particular, a method comprises receiving an input indicating an odorant, generating an odorant vector representing the odorant, generating an olfactory receptor vector, and determining one or more predicted olfactory percepts associated with the odorant based on the odorant vector and the olfactory receptor vector.

### CROSS-REFERENCE TO RELATED APPLICATION

This application claims the priority benefit of U.S. Provisional Patent Application No. 63/536,281, filed on Sep. 1, 2023, the entire disclosure of which is hereby incorporated by reference in its entirety.

### BACKGROUND

Humans use a family of more than 400 olfactory receptors to detect odors. When a person smells a morning coffee, a group of odorant molecules activate a combination of olfactory receptor (OR) proteins, transmitting a signal to the brain, which is processed and results in a novel percept. Deep learning has brought enormous gains in the digitization and understanding of vision and hearing. There are maps for color (e.g., RGB), and hearing (e.g., frequencies), but not for unique smells. Smell perception involves the detection and interpretation of thousands of chemical compounds present in the environment. The relationship between these compounds and the resulting smell perceptions is intricate and not easily reducible to a simple color-like representation since multiple molecules activate the same receptor and multiple olfactory receptors are activated by the same molecule.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

In accordance with examples of the present disclosure, an odor prediction tool allows users to predict perceptual responses of odorant molecules based on odorant molecular structures and odorant-olfactory receptor binding predictions.

In accordance with at least one example of the present disclosure, a method for predicting an odorant-receptor interaction is provided. The method includes receiving a first input indicating an odorant, determining molecular features of the odorant, receiving a second input indicating an olfactory receptor, determining protein features of the olfactory receptor, generating an odorant weight vector and an olfactory receptor weight vector based on the molecular features and the protein features, and determining a predicted interaction between the odorant and the olfactory receptor based on the odorant weight vector and the olfactory receptor weight vector.

In accordance with at least one example of the present disclosure, a method for determining predicted olfactory perception is provided. The method includes receiving an input indicating an odorant, generating an odorant vector representing the odorant, generating an olfactory receptor vector indicating which olfactory receptors of a plurality of olfactory receptors are predicted to be activated by the odorant, and determining one or more predicted olfactory percepts associated with the odorant based on the odorant vector and the olfactory receptor vector.

In accordance with at least one example of the present disclosure, a computing device for determining predicted olfactory perception is provided. The computing device may include a processor and a memory having a plurality of instructions stored thereon that, when executed by the processor, causes the computing device to receive an input indicating an odorant, generate an odorant vector representing the odorant, generate an olfactory receptor vector using an interaction prediction model, the olfactory receptor vector indicating which olfactory receptors of a plurality of olfactory receptors are predicted to be activated by the odorant, and determine one or more predicted olfactory percepts associated with the odorant based on the odorant vector and the olfactory receptor vector.

This Summary is provided to introduce a selection of concepts in a simplified form, which is further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the following description and, in part, will be apparent from the description, or may be learned by practice of the disclosure.

### BRIEF DESCRIPTION OF THE DRAWINGS

Non-limiting and non-exhaustive examples are described with reference to the following Figures.

FIG. 1 depicts a block diagram of an example of an operating environment in which an odorant prediction tool may be implemented in accordance with examples of the present disclosure;

FIG. 2 illustrates an example of olfactory perceptions predicted to be associated with an odorant in accordance with examples of the present disclosure;

FIG. 3 is a block diagram illustrating an exemplary overview of predicting a probability of an odorant-olfactory receptor (OR) interaction in accordance with examples of the present disclosure;

FIG. 4 is a block diagram illustrating an exemplary overview of determining predicted olfactory perception in accordance with examples of the present disclosure;

FIG. 5 depicts a flowchart of an example method for predicting a probability of an odorant-olfactory receptor (OR) interaction in accordance with examples of the present disclosure;

FIG. 6 depicts a flowchart of an example method for determining predicted olfactory perception in accordance with examples of the present disclosure;

FIGS. 7A and 7B illustrate overviews of an example generative machine learning model that may be used in accordance with examples of the present disclosure;

FIG. 8 is a block diagram illustrating example physical components of a computing device with which aspects of the disclosure may be practiced;

FIG. 9 is a simplified block diagram of a computing device with which aspects of the present disclosure may be practiced; and

FIG. 10 is a simplified block diagram of a distributed computing system in which aspects of the present disclosure may be practiced.

### DETAILED DESCRIPTION

In the following detailed description, references are made to the accompanying drawings that form a part hereof, and in which are shown by way of illustrations specific aspects or examples. These aspects may be combined, other aspects may be utilized, and structural changes may be made without departing from the present disclosure. Aspects may be practiced as methods, systems or devices. Accordingly, aspects may take the form of a hardware implementation, an entirely software implementation, or an implementation combining software and hardware aspects. The following detailed description is therefore not to be taken in a limiting sense, and the scope of the present disclosure is defined by the appended claims and their equivalents.

Humans use a family of more than 400 olfactory receptors to detect odors. When a person smells a morning coffee, a group of odorant molecules activate a combination of olfactory receptor (OR) proteins, transmitting a signal to the brain, which is processed and results in a novel percept. Deep learning has brought enormous gains in the digitization and understanding of vision and hearing. There are maps for color (e.g., RGB), and hearing (e.g., frequencies), but not for unique smells. Smell perception involves the detection and interpretation of thousands of chemical compounds present in the environment. The relationship between these compounds and the resulting smell perceptions is intricate and not easily reducible to a simple color-like representation since multiple molecules activate the same receptor and multiple olfactory receptors are activated by the same molecule (e.g., no 1-1 matching).

In accordance with examples of the present disclosure, an odor prediction tool is configured to predict olfactory perceptions associated with an odorant molecule based on the structure of the odorant molecule and one or more olfactory receptors that are likely to interact with the odorant molecule. The odor prediction tool may allow users to find a list of odorant molecules that are likely to smell like a desired scent or fragrance (e.g., one or more odor descriptors). For example, if a user wants to find a molecule that smells like vanilla that is creamy and sweet, the user may use the odor prediction tool to predict a list of candidate odorant molecules that are likely to smell like vanilla that is creamy and sweet. Additionally, the odor prediction tool may further allow users to design a new synthetic odorant to create a desired scent or fragrance.

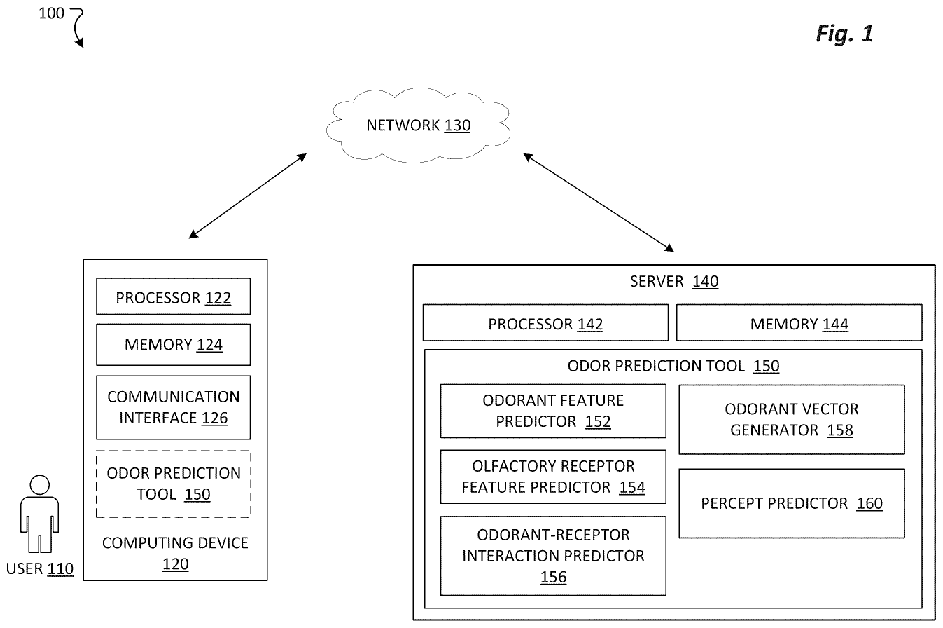

FIG. 1 depicts a block diagram of an example of an operating environment 100 in which an odor prediction tool may be implemented in accordance with examples of the present disclosure. To do so, the operating environment 100 includes a computing device 120 associated with the user 110. The operating environment 100 may further include one or more remote devices, such as a server 140, that are communicatively coupled to the computing device 120 via a network 130. The network 130 may include any kind of computing network including, without limitation, a wired or wireless local area network (LAN), a wired or wireless wide area network (WAN), and/or the Internet.

The computing device 120 includes a processor 122, a memory 124, and a communication interface 126. In some embodiments, the odor prediction tool 150 may be executed on the computing device 120. Additionally, the computing device 120 may be, but is not limited to, a computer, a notebook, a laptop, a mobile device, a smartphone, a tablet, a portable device, a wearable device, or any other suitable computing device that is capable of communicating with the server 140. The server 140 includes a processor 142, a memory 144, and an odor prediction tool 150. The server 140 may be any suitable computing device that is capable of executing the odor prediction tool 150.

The odor prediction tool 150 is configured to determine predicted olfactory perception. For example, the odor prediction tool 150 is configured to predict olfactory perceptions associated with an odorant molecule based on the structure of the odorant molecule and one or more olfactory receptors that are likely to interact with the odorant molecule. To do so, the odor prediction tool 150 further includes an odorant feature predictor 152, an olfactory receptor feature predictor 154, an odorant-receptor interaction predictor 156, an odorant vector generator 158, and a precept predictor 160.

The odorant feature predictor 152 is configured to determine molecular features of an odorant molecule. To do so, the odorant feature predictor 152 is configured to receive a graph representation of a chemical structure of the odorant molecule. The graph representation includes nodes and edges, where the nodes represent atoms of the odorant molecule and the edges represent bonds between the atoms of the odorant molecule. The odorant feature predictor 152 is configured to generate a two-dimensional tensor that represents a number of atoms of the odorant molecule across a number of dimensions (e.g., an embedding size). For example, the odorant feature predictor 152 includes a graph neural network (GNN). As described further below, the molecular features are used as a reference to prioritize atoms of the odorant molecule that are more likely to involve in an interaction with an olfactory receptor.

The olfactory receptor feature predictor 154 is configured to determine protein features of an olfactory receptor protein. To do so, the olfactory receptor feature predictor 154 is configured to receive a sequence of the olfactory receptor. For example, the olfactory receptor feature predictor 154 includes a protein language model (e.g., ESM-2). The olfactory receptor feature predictor 154 is trained and used to predict structures of proteins based on sequences of the proteins. The olfactory receptor feature predictor 154 is configured to generate a two-dimensional tensor that represents a number of residues of the olfactory receptor protein across a number of dimensions (e.g., an embedding size). As described further below, the protein features are used as a reference to prioritize residues of the olfactory receptor protein that are more likely to involve in an interaction with an odorant.

The odorant-receptor interaction predictor 156 is configured to determine a predicted interaction between an odorant molecule and an olfactory receptor protein upon obtaining two different inputs from two different modalities (e.g., one from the odorant molecule and one from the olfactory receptor protein). To do so, the odorant-receptor interaction predictor 156 is configured to generate an odorant weight vector and an olfactory receptor weight vector based on molecular features of the odorant molecule and protein features of the olfactory receptor. The odorant weight vector and the olfactory receptor weight vector are vectors of weights, each of which weigh the respective molecular and protein features (e.g., an order of importance of atoms of the odorant molecule and residues of the olfactory receptor, respectively).

The odorant-receptor interaction predictor 156 is configured to generate an odorant weight vector is a vector of weights representing the atoms of the odorant molecule in an order of importance when interacting with the olfactory receptor. To do so, the odorant-receptor interaction predictor 156 is configured to determine a weight for each atom of odorant molecule by prioritizing atoms that are more likely to interact with the olfactory receptor protein based on the molecular features and the protein features. For example, the weight assigned to each atom is a probability that the corresponding atom is likely to interact with the olfactory receptor.

The odorant-receptor interaction predictor 156 is further configured to generate an olfactory receptor weight vector is a vector of weights representing the residues of the olfactory receptor in an order of importance when interacting with the odorant. To do so, the odorant-receptor interaction predictor 156 is configured to determine a weight for each residue of olfactory receptor protein by prioritizing residues that are more likely to interact with the odorant molecule based on the molecular features and the protein features. For example, the weight assigned to each residue is a probability that the corresponding residue is likely to interact with the odorant.

Additionally, the odorant-receptor interaction predictor 156 is configured to determine a predicted interaction between the odorant molecule and the olfactory receptor protein based on the odorant weight vector and the olfactory receptor weight vector. To do so, the odorant-receptor interaction predictor 156 is configured to concatenate the odorant weight vector and the olfactory receptor weight vector and inputted into a multi-layer neural network (e.g., multiplayer perceptron (MLP) layers) to predict whether the odorant molecule interacts with the olfactory receptor. For example, the resulting prediction weight vector may be a probability that the selected odorant molecule interacts with the selected olfactory receptor.

Furthermore, the odorant-receptor interaction predictor 156 is configured to generate an olfactory receptor vector indicating which olfactory receptors are activated by the odorant. For example, the odorant-receptor interaction predictor 156 is trained to predict probabilities of interactions between odorants and olfactory receptors. Additionally, the odorant-receptor interaction predictor 156 is trained to determine importance of atoms of the selected odorant molecule and residues of the olfactory receptor when predicting a probability of whether the odorant molecule interacts with a particular olfactory receptor.

To do so, the odorant-receptor interaction predictor 156 is configured to receive sequences of preselected olfactory receptors and generate an olfactory receptor vector that indicates whether each of the olfactory receptors of the reselected olfactory receptors is predicted to be activated by the selected odorant molecule.

For example, the odorant-receptor interaction predictor 156 is configured to determine that an olfactory receptor protein is likely to be activated by the selected odorant molecule if a corresponding probability of interactions between the selected odorant and the corresponding olfactory receptor is above a predetermined threshold. For example, the predetermined threshold may be 50%. In such an example, if the predicted probability of interaction is above 50%, the interaction prediction model assigns “1” to the corresponding olfactory receptor. If the predicted probability of interaction is belove 50%, the interaction prediction model assigns “0” to the corresponding olfactory receptor. As such, the olfactory receptor vector indicates whether the olfactory receptors are activated by the selected odorant.

The odorant vector generator 158 is configured to generate an odorant vector representing an odorant molecule. For example, the odorant vector generator 158 is configured to convert a chemical structure of the selected odorant molecule into a graph representation (e.g., with nodes and edges) and generate an odorant vector. For example, the odorant vector is a 1×n vector that is generate by averaging a two-dimensional tensor, which represents a number of atoms of the selected odorant molecule across a number of dimensions (e.g., an embedding size).

The precept predictor 160 is configured to determine one or more predicted olfactory percepts associated with an odorant molecule based on an odorant vector associated with the odorant molecule and an olfactory receptor vector associated with an olfactory receptor. To do so, the precept predictor 160 is configured to receive the odorant vector and the olfactory receptor vector that are concatenated to generate a predicted output that indicates one or more odor descriptors that are associated with the odorant molecule. For example, the precept predictor 160 is a neural network.

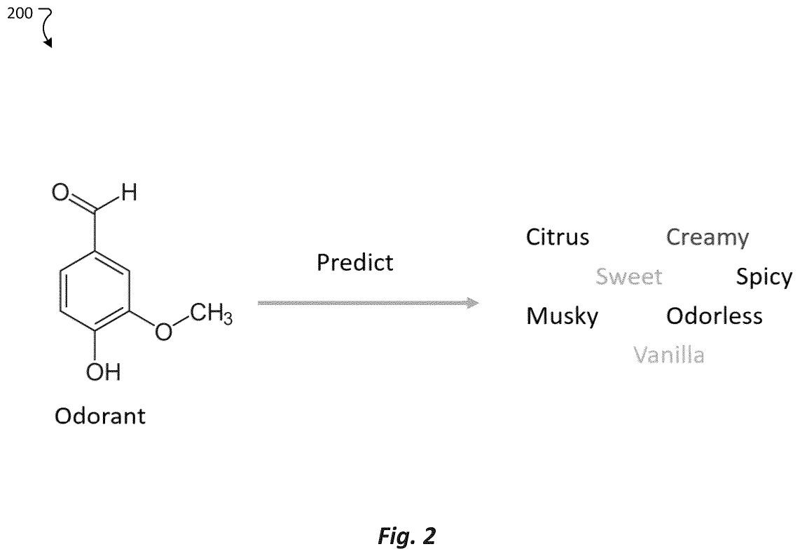

Referring now to FIG. 2, a hypothetical example 200 illustrates predicting olfactory perceptions (e.g., odor descriptors) from an odorant structure in accordance with examples of the present disclosure. As illustrated in FIG. 2, a chemical structure of the odorant molecule is converted into a graph representation with node information. The graph representation is passed through a graph neural network (GNN), which learns a new representation of nodes, and predict which subset of the different odor descriptor does the odorant molecule smell like based on the learned presentation. However, direct mapping between odorant structures and percepts is challenging because the olfactory percepts are not merely a function of the odorant molecules. For example, molecules with same odor profiles may be structurally dissimilar, and molecules that are structurally similar may have very different odor profiles.

As described above, in accordance with examples of the present disclosure, the odor prediction tool 150 is configured to predict olfactory perceptions associated with an odorant molecule based on the structure of the odorant molecule and one or more olfactory receptors that are likely to interact with the odorant molecule. In other words, the odor prediction tool 150 allows users to find a list of odorant molecules that are likely to smell like a desired scent or fragrance (e.g., one or more odor descriptors). For example, if a user wants to find a molecule that smells like vanilla that is creamy and sweet, the user may use the odor prediction tool 150 to predict a list of candidate odorant molecules that are likely to smell like vanilla that is creamy and sweet. Additionally, the odor prediction tool 150 further allows users to design a new synthetic odorant to create a desired scent or fragrance.

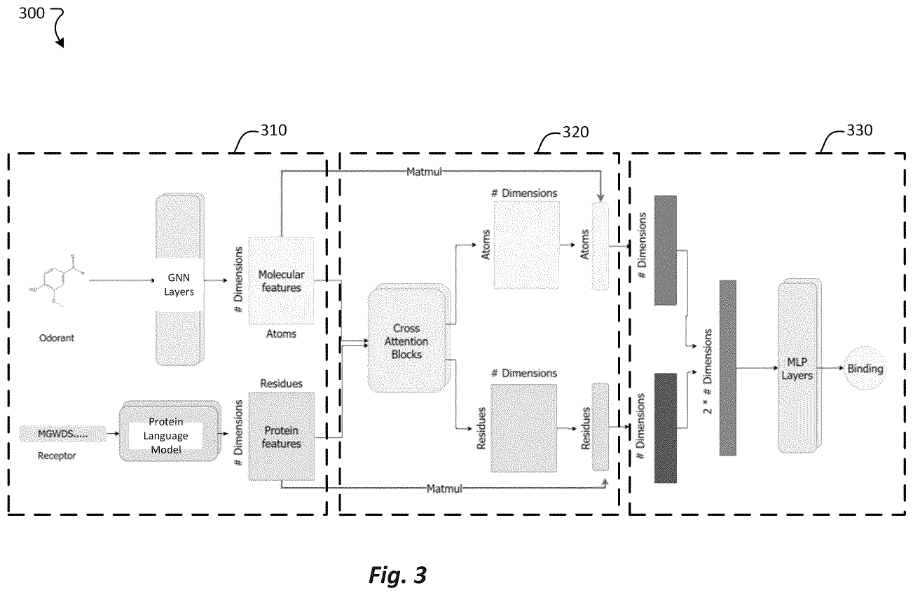

Referring now to FIG. 3, a block diagram 300 illustrates an exemplary overview of predicting a probability of an odorant-olfactory receptor (OR) interaction in accordance with examples of the present disclosure. At operation 310, the odor prediction tool 150 receives an input indicating a selected odorant molecule and a selected olfactory receptor. For example, a user may select an odorant molecule and an olfactory receptor to determine interaction between the selected odorant molecule and the selected olfactory receptor.

The odor prediction tool 150 further determines molecular features of the selected odorant molecule. For example, the molecular features of the selected odorant molecule are determined using a graph neural network (GNN). To do so, a chemical structure of the selected odorant molecule is converted into a graph representation (e.g., with nodes and edges) and fed into the GNN layers. The GNN generates a two-dimensional tensor that represents a number of atoms of the selected odorant molecule across a number of dimensions (e.g., an embedding size). As described further below, the molecular features are used as a reference to prioritize atoms of the selected odorant molecule that are more likely to involve in an interaction with a selected olfactory receptor.

The odor prediction tool 150 further determines protein features of the selected olfactory receptor protein. For example, the protein features of the selected olfactory receptor are determined using a protein language model (e.g., ESM-2). As described above, the protein language model is trained and used to predict structures of proteins based on sequences of the proteins. To do so, a sequence of the selected olfactory receptor is fed into the protein language model. Subsequently, the protein language model generates a two-dimensional tensor that represents a number of residues of the selected olfactory receptor protein across a number of dimensions (e.g., an embedding size). As described further below, the protein features are used as a reference to prioritize residues of the selected olfactory receptor protein that are more likely to involve in an interaction with the selected odorant.

At operation 320, the odor prediction tool 150 generates an odorant weight vector of weights and an olfactory receptor weight vector of weights based on the molecular features and the protein features. The odorant weight vector and the olfactory receptor weight vector represent an order of importance of atoms of the odorant molecule and residues of the olfactory receptor, respectively. For example, the odorant weight vector represents the atoms of the selected odorant molecule in an order of importance when interacting with the selected olfactory receptor. To do so, the odor prediction tool 150 determines a weight for each atom of selected odorant molecule by prioritizing atoms that are more likely to interact with the selected olfactory receptor protein based on the molecular features and the protein features. For example, the weight assigned to each atom is a probability that the corresponding atom is likely to interact with the selected olfactory receptor.

Similarly, the olfactory receptor weight vector represents the residues of the selected olfactory receptor in an order of importance when interacting with the selected odorant. To do so, the odor prediction tool 150 determines a weight for each residue of selected olfactory receptor protein by prioritizing residues that are more likely to interact with the selected odorant molecule based on the molecular features and the protein features. For example, the weight assigned to each residue is a probability that the corresponding residue is likely to interact with the selected odorant.

At operation 330, the odor prediction tool 150 determines a predicted interaction between the selected odorant molecule and the selected olfactory receptor protein based on the odorant weight vector and the olfactory receptor weight vector. To do so, the odorant weight vector and the olfactory receptor weight vector are concatenated and fed into a multi-layer neural network (e.g., multiplayer perceptron (MLP) layers) to predict whether the selected odorant molecule interacts with the selected olfactory receptor. For example, the resulting prediction weight vector may be a probability that the selected odorant molecule interacts with the selected olfactory receptor.

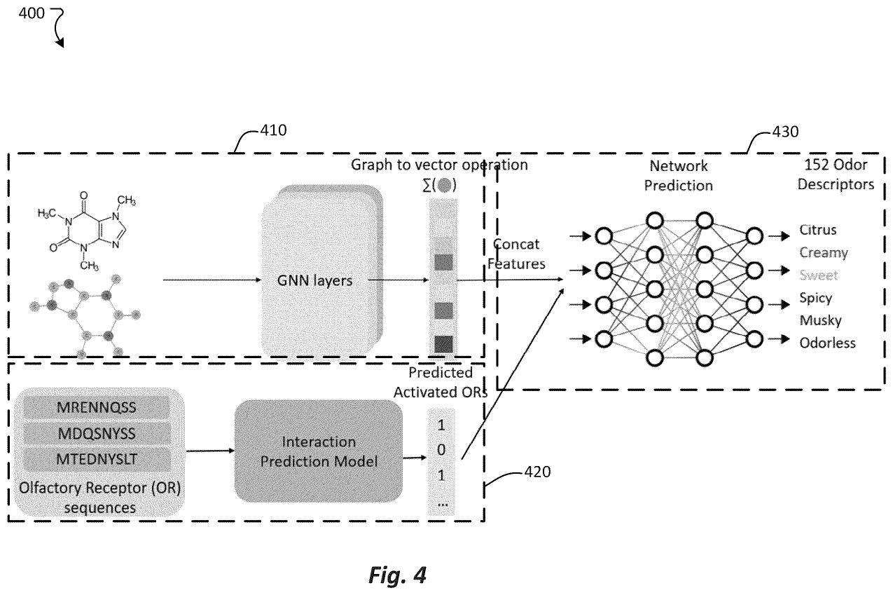

Referring now to FIG. 4, a block diagram 400 illustrates an exemplary overview of determining predicted olfactory perception in accordance with examples of the present disclosure.

As described further below, at operation 410, the odor prediction tool 150 receives an indication of a selected odorant molecule. For example, the selected odorant molecule is a target odorant that a user has selected to determine predicted olfactory percepts (e.g., one or more odor descriptors) associated with the selected odorant molecule. Subsequently, the odor prediction tool 150 generates an odorant vector representing the selected odorant molecule. As illustrated in FIG. 4, a chemical structure of the selected odorant molecule is converted into a graph representation (e.g., with nodes and edges) and fed into graph neural network (GNN) layers. The GNN generates an odorant vector, which is a vector representation of the selected odorant molecule. For example, to do so, a two-dimensional tensor that represents a number of atoms of the selected odorant molecule across a number of dimensions (e.g., an embedding size) is generated and simplified into a lxn vector by averaging the two-dimensional tensor.

At operation 420, the odor prediction tool 150 generates an olfactory receptor vector indicating which olfactory receptors are activated by the selected odorant using an interaction prediction model. As described above, the interaction prediction model is trained to predict probabilities of interactions between odorants and olfactory receptors. Additionally, the interaction prediction model is trained to determine importance of atoms of the selected odorant molecule and residues of the olfactory receptor when predicting a probability of whether the selected odorant molecule interacts with a particular olfactory receptor.

As illustrated in FIG. 4, sequences of preselected olfactory receptors are passed through the interaction prediction model. The interaction prediction model generates an olfactory receptor vector that indicates whether each of the olfactory receptors is predicted to be activated by the selected odorant molecule. For example, the interaction prediction model determines that an olfactory receptor protein is likely to be activated by the selected odorant molecule if a corresponding probability of interactions between the selected odorant and the corresponding olfactory receptor is above a predetermined threshold. For example, the predetermined threshold may be 50%. In such an example, if the predicted probability of interaction is above 50%, the interaction prediction model assigns “1” to the corresponding olfactory receptor. If the predicted probability of interaction is below 50%, the interaction prediction model assigns “0” to the corresponding olfactory receptor. As such, the olfactory receptor vector indicates whether the olfactory receptors are activated by the selected odorant.

At operation 430, the odor prediction tool 150 determines one or more predicted olfactory percepts associated with the selected odorant molecule based on the odorant vector and the olfactory receptor vector. To do so, for example, the odorant vector and the olfactory receptor vector are concatenated and fed into a neural network to generate a predicted output that indicates one or more odor descriptors that are associated with the selected odorant molecule.

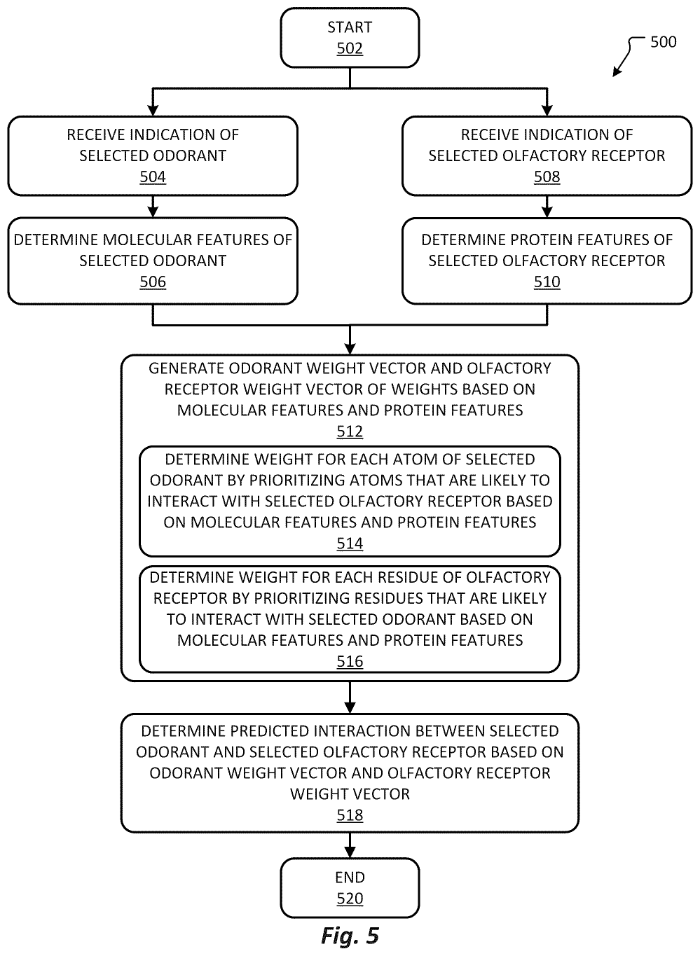

Referring now to FIG. 5, a method 500 for predicting an odorant-receptor interaction is provided. A general order for the steps of the method 500 is shown in FIG. 5. Generally, the method 500 starts at 502 and ends at 520. The method 500 may include more or fewer steps or may arrange the order of the steps differently than those shown in FIG. 5. In the illustrative aspect, the method 500 is performed by a computing device (e.g., a server 140). However, it should be appreciated that one or more steps of the method 500 may be performed by another device (e.g., a user device 120) of a user 110.

Specifically, in some aspects, the method 500 may be performed by an odor prediction tool (e.g., 150) executed on the server 140. For example, the server 140 may be any suitable computing device that is capable of executing an odor prediction tool (e.g., 150). For example, the computing device 120 may be, but is not limited to, a computer, a notebook, a laptop, a mobile device, a smartphone, a tablet, a portable device, a wearable device, or any other suitable computing device that is capable of communicating with the server (e.g., 140). The method 500 can be executed as a set of computer-executable instructions executed by a computer system and encoded or stored on a computer readable medium. Further, the method 500 can be performed by gates or circuits associated with a processor, Application Specific Integrated Circuit (ASIC), a field programmable gate array (FPGA), a system on chip (SOC), or other hardware device. Hereinafter, the method 500 shall be explained with reference to the systems, components, modules, software, data structures, user interfaces, etc. described in conjunction with FIG. 1 and FIGS. 7-10.

The method 500 starts at operation 502, where flow may proceed to operation 504. At operation 504, the odor prediction tool 150 receives an indication of a selected odorant molecule. For example, the selected odorant molecule is a target odorant that a user has selected to determine interaction with one or more olfactory receptors (e.g., a selected olfactory receptor in operation 508).

At operation 506, the odor prediction tool 150 determines molecular features of the selected odorant molecule. For example, as shown in FIG. 3, the molecular features of the selected odorant molecule are determined using a graph neural network (GNN). To do so, a chemical structure of the selected odorant molecule is converted into a graph representation (e.g., with nodes and edges) and fed into the GNN layers. The GNN generates a two-dimensional tensor that represents a number of atoms of the selected odorant molecule across a number of dimensions (e.g., an embedding size). As described further below, the molecular features are used as a reference to prioritize atoms of the selected odorant molecule that are more likely to involve in an interaction with a selected olfactory receptor.

At operation 508, the odor prediction tool 150 receives an indication of a selected olfactory receptor protein. For example, the selected olfactory receptor protein is a target olfactory receptor that the user has selected to determine interaction with one or more odorant molecules (e.g., the selected odorant molecule).

At operation 510, the odor prediction tool 150 determines protein features of the selected olfactory receptor protein. For example, as shown in FIG. 3, the protein features of the selected olfactory receptor are determined using a protein language model (e.g., ESM-2). As described above, the protein language model is trained and used to predict structures of proteins based on sequences of the proteins. To do so, a sequence of the selected olfactory receptor is fed into the protein language model. Subsequently, the protein language model generates a two-dimensional tensor that represents a number of residues of the selected olfactory receptor protein across a number of dimensions (e.g., an embedding size). As described further below, the protein features are used as a reference to prioritize residues of the selected olfactory receptor protein that are more likely to involve in an interaction with the selected odorant.

At operation 512, upon obtaining two different inputs from two different modalities (e.g., one from the odorant molecule and one from the olfactory receptor protein), the odor prediction tool 150 generates an odorant weight vector of weights and an olfactory receptor weight vector of weights based on the molecular features and the protein features. The odorant weight vector and the olfactory receptor weight vector represent an order of importance of atoms of the odorant molecule and residues of the olfactory receptor, respectively.

For example, the odorant weight vector represents the atoms of the selected odorant molecule in an order of importance when interacting with the selected olfactory receptor. To do so, at operation 514, the odor prediction tool 150 determines a weight for each atom of selected odorant molecule by prioritizing atoms that are more likely to interact with the selected olfactory receptor protein based on the molecular features and the protein features. For example, the weight assigned to each atom is a probability that the corresponding atom is likely to interact with the selected olfactory receptor.

Similarly, the olfactory receptor weight vector represents the residues of the selected olfactory receptor in an order of importance when interacting with the selected odorant. To do so, at operation 516, the odor prediction tool 150 determines a weight for each residue of selected olfactory receptor protein by prioritizing residues that are more likely to interact with the selected odorant molecule based on the molecular features and the protein features. For example, the weight assigned to each residue is a probability that the corresponding residue is likely to interact with the selected odorant.

Subsequently, at operation 518, the odor prediction tool 150 determines a predicted interaction between the selected odorant molecule and the selected olfactory receptor protein based on the odorant weight vector and the olfactory receptor weight vector. To do so, as illustrated in FIG. 3, the odorant weight vector and the olfactory receptor weight vector are concatenated and fed into a multi-layer neural network (e.g., multiplayer perceptron (MLP) layers) to predict whether the selected odorant molecule interacts with the selected olfactory receptor. For example, the resulting prediction output may be a probability that the selected odorant molecule interacts with the selected olfactory receptor.

The method 500 may end at operation 520. However, it should be appreciated that, although it is not shown in FIG. 5, the method 500 (i.e., operation 508-518) may be repeated for each olfactory receptor of a plurality of preselected olfactory receptors to determine a predicted probability of the corresponding olfactory receptor interacting with the selected odorant molecule. Such results (e.g., in a vector form) associated with the selected odorant may be stored in a database. In certain embodiments, the method 500 may further be repeated for a plurality of preselected odorants with the plurality of preselected olfactory receptors.

In some embodiments, the predicted interactions between odorants and the olfactory receptors are stored in a database. The database may be used to determine one or more odorants that are predicted to be associated a desired olfactory percept. For example, a desired olfactory percept may be received from a user, and one or more odorants that are predicted to be associated with the desired olfactory percept are determined based at least in part on the database. A list of the one or more odorants may be presented to the user.

It should be appreciated that, in some embodiments, the operations 504-506 may be performed prior to the operations 508-510. Alternatively, in certain embodiments, the operations 504-506 may be performed subsequent to the operations 508-510. In some embodiments, the operations 504-506 may be performed concurrently with the operations 508-510.

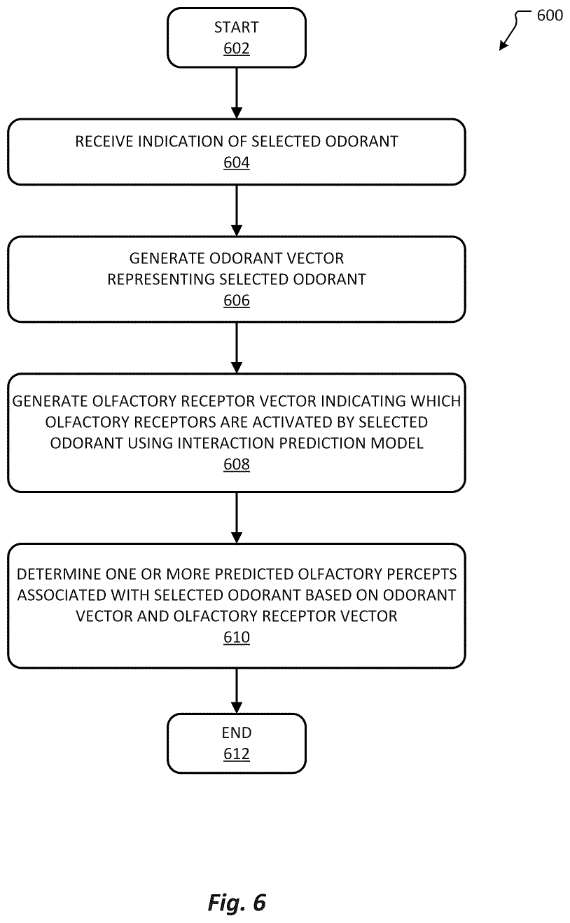

Referring now to FIG. 6, a method 600 for determining predicted olfactory perception is provided. A general order for the steps of the method 600 is shown in FIG. 6. Generally, the method 600 starts at 602 and ends at 612. The method 600 may include more or fewer steps or may arrange the order of the steps differently than those shown in FIG. 6. In the illustrative aspect, the method 600 is performed by a computing device (e.g., a server 140). However, it should be appreciated that one or more steps of the method 600 may be performed by another device (e.g., a user device 120) of a user 110.

Specifically, in some aspects, the method 600 may be performed by an odor prediction tool (e.g., 150) executed on the server 140. For example, the server 140 may be any suitable computing device that is capable of executing an odor prediction tool (e.g., 150). For example, the computing device 120 may be, but is not limited to, a computer, a notebook, a laptop, a mobile device, a smartphone, a tablet, a portable device, a wearable device, or any other suitable computing device that is capable of communicating with the server (e.g., 140). The method 600 can be executed as a set of computer-executable instructions executed by a computer system and encoded or stored on a computer readable medium. Further, the method 600 can be performed by gates or circuits associated with a processor, Application Specific Integrated Circuit (ASIC), a field programmable gate array (FPGA), a system on chip (SOC), or other hardware device. Hereinafter, the method 600 shall be explained with reference to the systems, components, modules, software, data structures, user interfaces, etc. described in conjunction with FIG. 1 and FIGS. 7-10.

The method 600 starts at operation 602, where flow may proceed to operation 604. At operation 604, the odor prediction tool 150 receives an indication of a selected odorant molecule. For example, the selected odorant molecule is a target odorant that a user has selected to determine predicted olfactory percepts (e.g., one or more odor descriptors) associated with the selected odorant molecule.

At operation 606, the odor prediction tool 150 generates an odorant vector representing the selected odorant molecule. For example, as illustrated in FIG. 4, a chemical structure of the selected odorant molecule is converted into a graph representation (e.g., with nodes and edges) and fed into graph neural network (GNN) layers. The GNN generates an odorant vector, which is a vector representation of the selected odorant molecule. For example, to do so, a two-dimensional tensor that represents a number of atoms of the selected odorant molecule across a number of dimensions (e.g., an embedding size) is generated and simplified into a 1×n vector by averaging the two-dimensional tensor.

At operation 608, the odor prediction tool 150 generates an olfactory receptor vector indicating which olfactory receptors are activated by the selected odorant using an interaction prediction model. As described above in FIGS. 3 and 5, the interaction prediction model is trained to predict probabilities of interactions between odorants and olfactory receptors. Additionally, the interaction prediction model is trained to determine importance of atoms of the selected odorant molecule and residues of the olfactory receptor when predicting a probability of whether the selected odorant molecule interacts with a particular olfactory receptor.

In some embodiments, as illustrated in FIG. 4, sequences of preselected olfactory receptors are passed through the interaction prediction model. The interaction prediction model generates an output vector that indicates whether each of the olfactory receptors is predicted to be activated by the selected odorant molecule. For example, the interaction prediction model determines that an olfactory receptor protein is likely to be activated by the selected odorant molecule if a corresponding probability of interactions between the selected odorant and the corresponding olfactory receptor is above a predetermined threshold. For example, the predetermined threshold may be 50%. In such an example, if the predicted probability of interaction is above 50%, the interaction prediction model assigns “1” to the corresponding olfactory receptor. If the predicted probability of interaction is belove 50%, the interaction prediction model assigns “0” to the corresponding olfactory receptor. As such, the olfactory receptor vector indicates whether the olfactory receptors are activated by the selected odorant.

At operation 610, the odor prediction tool 150 determines one or more predicted olfactory percepts (e.g., odor descriptors in FIG. 2) associated with the selected odorant molecule based on the odorant vector and the olfactory receptor vector. To do so, for example, the odorant vector and the olfactory receptor vector are concatenated and fed into a neural network to generate a predicted output that indicates one or more odor descriptors that are associated with the selected odorant molecule.

In some embodiments, a mapping between odorant and one or more olfactory receptors predicted to be activated by the odorant and the one or more predicted olfactory percepts are stored in a database. The database may be used to determine one or more odorants that are predicted to be associated a desired olfactory percept. For example, a desired olfactory percept may be received from a user, and one or more odorants that are predicted to be associated with the desired olfactory percept are determined based at least in part on the database. A list of the one or more odorants may be presented to the user. The method 600 may end at 612.

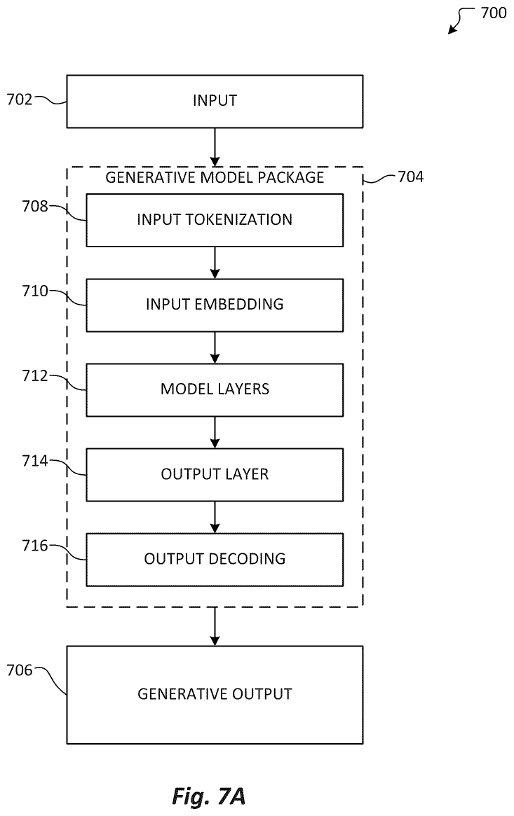

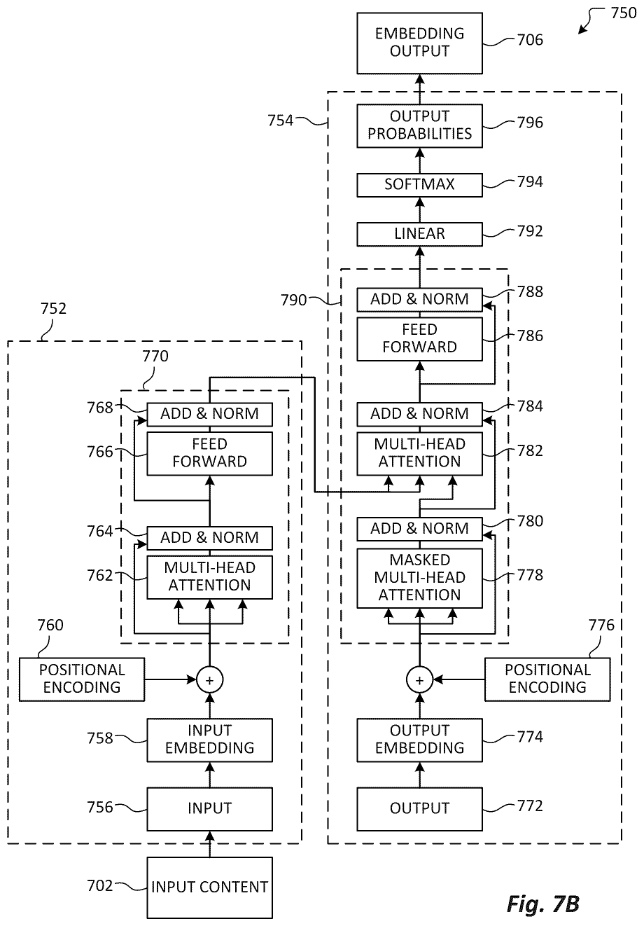

FIGS. 7A and 7B illustrate overviews of an example generative machine learning model that may be used according to aspects described herein. With reference first to FIG. 7A, conceptual diagram 700 depicts an overview of pre-trained generative model package 704 that processes an input 702 to generate model output for storing entries in and/or retrieving information from a generative model output 706 (e.g., a structured output) according to aspects described herein.

In examples, generative model package 704 is pre-trained according to a variety of inputs (e.g., a variety of human languages, a variety of programming languages, and/or a variety of content types) and therefore need not be finetuned or trained for a specific scenario. Rather, generative model package 704 may be more generally pre-trained, such that input 702 includes a prompt that is generated, selected, or otherwise engineered to induce generative model package 704 to produce certain generative model output 706. It will be appreciated that input 702 and generative model output 706 may each include any of a variety of content types, including, but not limited to, text output, image output, audio output, video output, programmatic output, and/or binary output, among other examples. In examples, input 702 and generative model output 706 may have different content types, as may be the case when generative model package 704 includes a generative multimodal machine learning model.

As such, generative model package 704 may be used in any of a variety of scenarios and, further, a different generative model package may be used in place of generative model package 704 without substantially modifying other associated aspects (e.g., similar to those described herein with respect to FIGS. 1-3). Accordingly, generative model package 704 operates as a tool with which machine learning processing is performed, in which certain inputs 702 to generative model package 704 are programmatically generated or otherwise determined, thereby causing generative model package 704 to produce model output 706 that may subsequently be used for further processing.

Generative model package 704 may be provided or otherwise used according to any of a variety of paradigms. For example, generative model package 704 may be used local to a computing device (e.g., the computing device 120 in FIG. 1) or may be accessed remotely from a machine learning service (e.g., the server 140 in FIG. 1). In other examples, aspects of generative model package 704 are distributed across multiple computing devices. In some instances, generative model package 704 is accessible via an application programming interface (API), as may be provided by an operating system of the computing device and/or by the machine learning service, among other examples.

With reference now to the illustrated aspects of generative model package 704, generative model package 704 includes input tokenization 708, input embedding 710, model layers 712, output layer 714, and output decoding 716. In examples, input tokenization 708 processes input 702 to generate input embedding 710, which includes a sequence of symbol representations that corresponds to input 702. Accordingly, input embedding 710 is processed by model layers 712, output layer 714, and output decoding 716 to produce model output 706. An example architecture corresponding to generative model package 704 is depicted in FIG. 7B, which is discussed below in further detail. Even so, it will be appreciated that the architectures that are illustrated and described herein are not to be taken in a limiting sense and, in other examples, any of a variety of other architectures may be used.

FIG. 7B is a conceptual diagram that depicts an example architecture 750 of a pre-trained generative machine learning model that may be used according to aspects described herein. As noted above, any of a variety of alternative architectures and corresponding ML models may be used in other examples without departing from the aspects described herein.

As illustrated, architecture 750 processes input 702 to produce generative model output 706, aspects of which were discussed above with respect to FIG. 7A. Architecture 750 is depicted as a transformer model that includes encoder 752 and decoder 754. Encoder 752 processes input embedding 758 (aspects of which may be similar to input embedding 710 in FIG. 7A), which includes a sequence of symbol representations that corresponds to input 756. In examples, input 756 includes content data 702 corresponding to a content item.

Further, positional encoding 760 may introduce information about the relative and/or absolute position for tokens of input embedding 758. Similarly, output embedding 774 includes a sequence of symbol representations that correspond to output 772, while positional encoding 776 may similarly introduce information about the relative and/or absolute position for tokens of output embedding 774.

As illustrated, encoder 752 includes example layer 770. It will be appreciated that any number of such layers may be used, and that the depicted architecture is simplified for illustrative purposes. Example layer 770 includes two sub-layers: multi-head attention layer 762 and feed forward layer 766. In examples, a residual connection is included around each layer 762, 766, after which normalization layers 764 and 768, respectively, are included.

Decoder 754 includes example layer 790. Similar to encoder 752, any number of such layers may be used in other examples, and the depicted architecture of decoder 754 is simplified for illustrative purposes. As illustrated, example layer 790 includes three sub-layers: masked multi-head attention layer 778, multi-head attention layer 782, and feed forward layer 786. Aspects of multi-head attention layer 782 and feed forward layer 786 may be similar to those discussed above with respect to multi-head attention layer 762 and feed forward layer 766, respectively. Additionally, masked multi-head attention layer 778 performs multi-head attention over the output of encoder 752 (e.g., output 772). In examples, masked multi-head attention layer 778 prevents positions from attending to subsequent positions. Such masking, combined with offsetting the embeddings (e.g., by one position, as illustrated by multi-head attention layer 782), may ensure that a prediction for a given position depends on known output for one or more positions that are less than the given position. As illustrated, residual connections are also included around layers 778, 782, and 786, after which normalization layers 780, 784, and 788, respectively, are included.

Multi-head attention layers 762, 778, and 782 may each linearly project queries, keys, and values using a set of linear projections to a corresponding dimension. Each linear projection may be processed using an attention function (e.g., dot-product or additive attention), thereby yielding n-dimensional output values for each linear projection. The resulting values may be concatenated and once again projected, such that the values are subsequently processed as illustrated in FIG. 7B (e.g., by a corresponding normalization layer 764, 780, or 784).

Feed forward layers 766 and 786 may each be a fully connected feed-forward network, which applies to each position. In examples, feed forward layers 766 and 786 each include a plurality of linear transformations with a rectified linear unit activation in between. In examples, each linear transformation is the same across different positions, while different parameters may be used as compared to other linear transformations of the feed-forward network.

Additionally, aspects of linear transformation 792 may be similar to the linear transformations discussed above with respect to multi-head attention layers 762, 778, and 782, as well as feed forward layers 766 and 786. Softmax 794 may further convert the output of linear transformation 792 to predicted next-token probabilities, as indicated by output probabilities 796. It will be appreciated that the illustrated architecture is provided in as an example and, in other examples, any of a variety of other model architectures may be used in accordance with the disclosed aspects.

Accordingly, output probabilities 796 may thus form generative model output 706 according to aspects described herein, such that the output of the generative ML model (e.g., which may include one or more state labels) is used as input for determining an action according to aspects described herein. In other examples, generative model output 706 is provided as generated structured output.

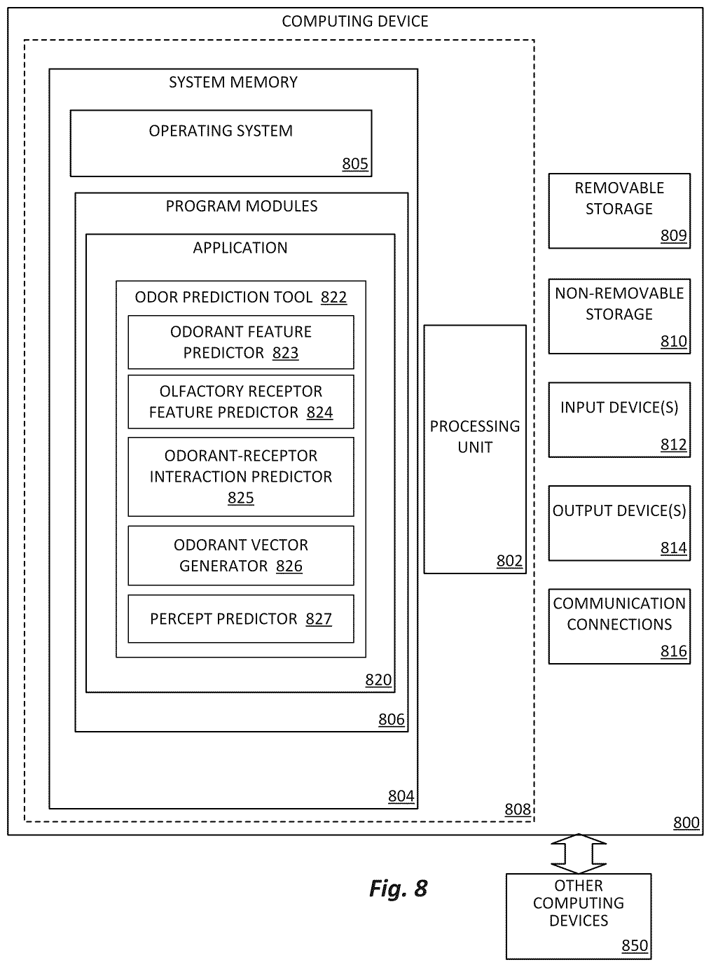

FIGS. 8-10 and the associated descriptions provide a discussion of a variety of operating environments in which aspects of the disclosure may be practiced. However, the devices and systems illustrated and discussed with respect to FIGS. 8-10 are for purposes of example and illustration and are not limiting of a vast number of computing device configurations that may be utilized for practicing aspects of the disclosure, described herein.

FIG. 8 is a block diagram illustrating physical components (e.g., hardware) of a computing device 800 with which aspects of the disclosure may be practiced. The computing device components described below may be suitable for the computing devices described above, including one or more devices associated with machine learning service (e.g., server 140), as well as computing device 120 discussed above with respect to FIG. 1. In a basic configuration, the computing device 800 may include at least one processing unit 802 and a system memory 804. Depending on the configuration and type of computing device, the system memory 804 may comprise, but is not limited to, volatile storage (e.g., random access memory), non-volatile storage (e.g., read-only memory), flash memory, or any combination of such memories.

The system memory 804 may include an operating system 805 and one or more program modules 806 suitable for running software application 820, such as one or more components supported by the systems described herein. As examples, system memory 804 may store an odor prediction tool 822, including an odorant feature predictor 823, an olfactory receptor feature predictor 824, an odorant-receptor interaction predictor 825, an odorant vector generator 826, and a percept predictor 827. The operating system 805, for example, may be suitable for controlling the operation of the computing device 800.

Furthermore, aspects of the disclosure may be practiced in conjunction with a graphics library, other operating systems, or any other application program and is not limited to any particular application or system. This basic configuration is illustrated in FIG. 8 by those components within a dashed line 808. The computing device 800 may have additional features or functionality. For example, the computing device 800 may also include additional data storage devices (removable and/or non-removable) such as, for example, magnetic disks, optical disks, or tape. Such additional storage is illustrated in FIG. 8 by a removable storage device 809 and a non-removable storage device 810.

As stated above, a number of program modules and data files may be stored in the system memory 804. While executing on the processing unit 802, the program modules 806 (e.g., application 820) may perform processes including, but not limited to, the aspects, as described herein. Other program modules that may be used in accordance with aspects of the present disclosure may include electronic mail and contacts applications, word processing applications, spreadsheet applications, database applications, slide presentation applications, drawing or computer-aided application programs, etc.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 8 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 800 on the single integrated circuit (chip). Aspects of the disclosure may also be practiced using other technologies capable of performing logical operations such as, for example, AND, OR, and NOT, including but not limited to mechanical, optical, fluidic, and quantum technologies. In addition, aspects of the disclosure may be practiced within a general purpose computer or in any other circuits or systems.

The computing device 800 may also have one or more input device(s) 812 such as a keyboard, a mouse, a pen, a sound or voice input device, a touch or swipe input device, etc. The output device(s) 814 such as a display, speakers, a printer, etc. may also be included. The aforementioned devices are examples and others may be used. The computing device 800 may include one or more communication connections 816 allowing communications with other computing devices 850. Examples of suitable communication connections 816 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 804, the removable storage device 809, and the non-removable storage device 810 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 800. Any such computer storage media may be part of the computing device 800. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

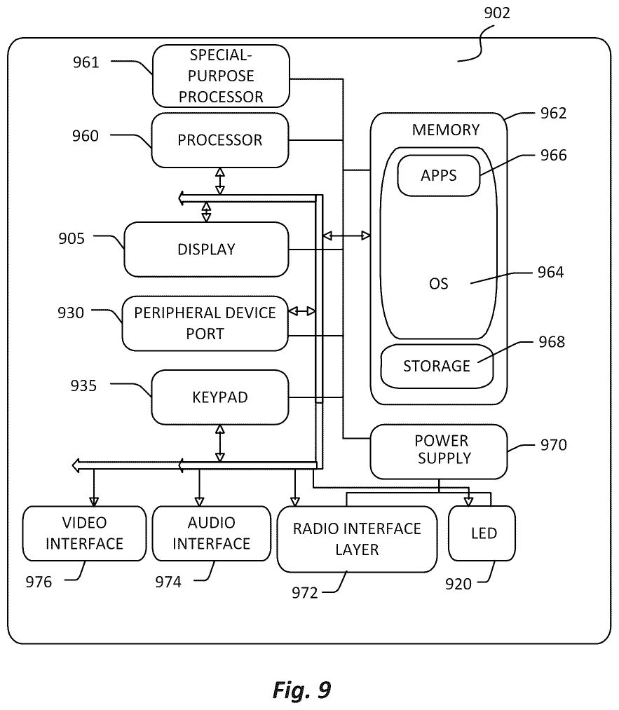

FIG. 9 illustrates a system 900 that may, for example, be a mobile computing device, such as a mobile telephone, a smart phone, wearable computer (such as a smart watch), a tablet computer, a laptop computer, and the like, with which aspects of the disclosure may be practiced. In one example, the system 900 is implemented as a “smart phone” capable of running one or more applications (e.g., browser, e-mail, calendaring, contact managers, messaging clients, games, and media clients/players). In some aspects, the system 900 is integrated as a computing device, such as an integrated personal digital assistant (PDA) and wireless phone.

In a basic configuration, such a mobile computing device is a handheld computer having both input elements and output elements. The system 900 typically includes a display 905 and one or more input buttons that allow the user to enter information into the system 900. The display 905 may also function as an input device (e.g., a touch screen display).

If included, an optional side input element allows further user input. For example, the side input element may be a rotary switch, a button, or any other type of manual input element. In alternative aspects, system 900 may incorporate more or less input elements. For example, the display 905 may not be a touch screen in some aspects. In another example, an optional keypad 935 may also be included, which may be a physical keypad or a “soft” keypad generated on the touch screen display.

In various aspects, the output elements include the display 905 for showing a graphical user interface (GUI), a visual indicator (e.g., a light emitting diode 920), and/or an audio transducer 925 (e.g., a speaker). In some aspects, a vibration transducer is included for providing the user with tactile feedback. In yet another aspect, input and/or output ports are included, such as an audio input (e.g., a microphone jack), an audio output (e.g., a headphone jack), and a video output (e.g., a HDMI port) for sending signals to or receiving signals from an external device.

One or more application programs 966 may be loaded into the memory 962 and run on or in association with the operating system 964. Examples of the application programs include phone dialer programs, e-mail programs, personal information management (PIM) programs, word processing programs, spreadsheet programs, Internet browser programs, messaging programs, and so forth. The system 900 also includes a non-volatile storage area 968 within the memory 962. The non-volatile storage area 968 may be used to store persistent information that should not be lost if the system 900 is powered down. The application programs 966 may use and store information in the non-volatile storage area 968, such as e-mail or other messages used by an e-mail application, and the like. A synchronization application (not shown) also resides on the system 900 and is programmed to interact with a corresponding synchronization application resident on a host computer to keep the information stored in the non-volatile storage area 968 synchronized with corresponding information stored at the host computer. As should be appreciated, other applications may be loaded into the memory 962 and run on the system 900 described herein (e.g., a content capture manager, a content retrieval manager, etc.).

The system 900 has a power supply 970, which may be implemented as one or more batteries. The power supply 970 might further include an external power source, such as an AC adapter or a powered docking cradle that supplements or recharges the batteries.

The system 900 may also include a radio interface layer 972 that performs the function of transmitting and receiving radio frequency communications. The radio interface layer 972 facilitates wireless connectivity between the system 900 and the “outside world,” via a communications carrier or service provider. Transmissions to and from the radio interface layer 972 are conducted under control of the operating system 964. In other words, communications received by the radio interface layer 972 may be disseminated to the application programs 966 via the operating system 964, and vice versa.

The visual indicator 920 may be used to provide visual notifications, and/or an audio interface 974 may be used for producing audible notifications via the audio transducer 925. In the illustrated example, the visual indicator 920 is a light emitting diode (LED) and the audio transducer 925 is a speaker. These devices may be directly coupled to the power supply 970 so that when activated, they remain on for a duration dictated by the notification mechanism even though the processor 960 and other components might shut down for conserving battery power. The LED may be programmed to remain on indefinitely until the user takes action to indicate the powered-on status of the device. The audio interface 974 is used to provide audible signals to and receive audible signals from the user. For example, in addition to being coupled to the audio transducer 925, the audio interface 974 may also be coupled to a microphone to receive audible input, such as to facilitate a telephone conversation. In accordance with aspects of the present disclosure, the microphone may also serve as an audio sensor to facilitate control of notifications, as will be described below. The system 900 may further include a video interface 976 that enables an operation of an on-board camera 930 to record still images, video stream, and the like.

It will be appreciated that system 900 may have additional features or functionality. For example, system 900 may also include additional data storage devices (removable and/or non-removable) such as, magnetic disks, optical disks, or tape. Such additional storage is illustrated in FIG. 9 by the non-volatile storage area 968.

Data/information generated or captured and stored via the system 900 may be stored locally, as described above, or the data may be stored on any number of storage media that may be accessed by the device via the radio interface layer 972 or via a wired connection between the system 900 and a separate computing device associated with the system 900, for example, a server computer in a distributed computing network, such as the Internet. As should be appreciated, such data/information may be accessed via the radio interface layer 972 or via a distributed computing network. Similarly, such data/information may be readily transferred between computing devices for storage and use according to any of a variety of data/information transfer and storage means, including electronic mail and collaborative data/information sharing systems.

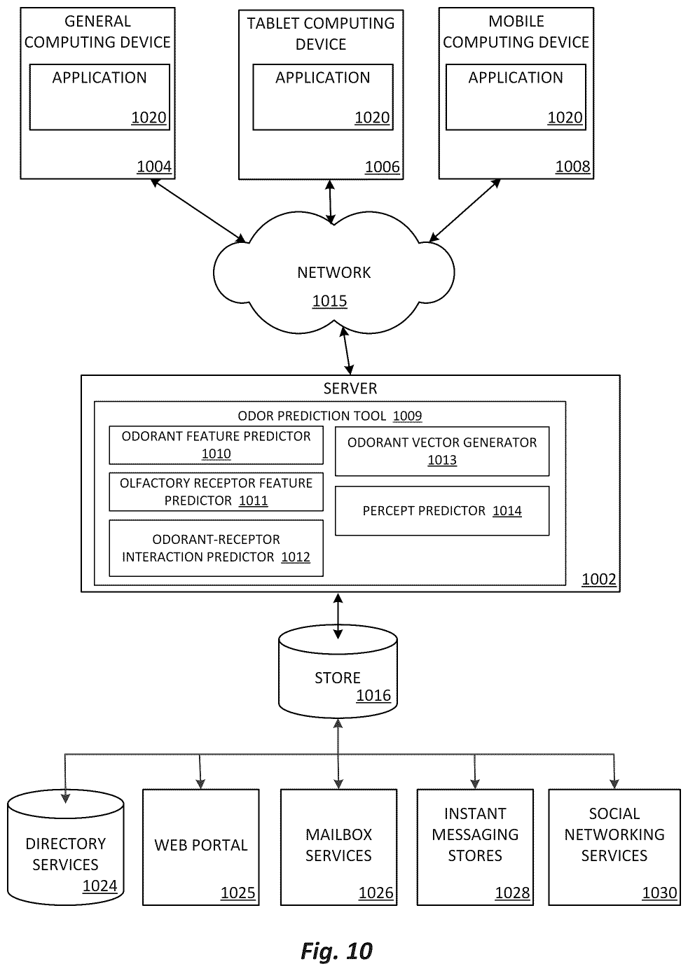

FIG. 10 illustrates one aspect of the architecture of a system for processing data received at a computing system from a remote source, such as a personal computer 1004, tablet computing device 1006, or mobile computing device 1008, as described above. Content displayed at server device 1002 may be stored in different communication channels or other storage types. For example, various documents may be stored using a directory service 1024, a web portal 1025, a mailbox service 1026, an instant messaging store 1028, or a social networking site 1030.

An application 1020 (e.g., similar to the application 520) may be employed by a client that communicates with server device 1002. Additionally, or alternatively, an odor prediction tool 1009, which includes an odorant feature predictor 1010, an olfactory receptor feature predictor 1011, an odorant-receptor interaction predictor 1012, an odorant vector generator 1013, and a percept predictor 1014, may be employed by server device 1002. The server device 1002 may provide data to and from a client computing device such as a personal computer 1004, a tablet computing device 1006 and/or a mobile computing device 1008 (e.g., a smart phone) through a network 1015. By way of example, the computer system described above may be embodied in a personal computer 1004, a tablet computing device 1006 and/or a mobile computing device 1008 (e.g., a smart phone). Any of these examples of the computing devices may obtain content from the store 1016, in addition to receiving graphical data useable to be either pre-processed at a graphic-originating system, or post-processed at a receiving computing system.

It will be appreciated that the aspects and functionalities described herein may operate over distributed systems (e.g., cloud-based computing systems), where application functionality, memory, data storage and retrieval and various processing functions may be operated remotely from each other over a distributed computing network, such as the Internet or an intranet. User interfaces and information of various types may be displayed via on-board computing device displays or via remote display units associated with one or more computing devices. For example, user interfaces and information of various types may be displayed and interacted with on a wall surface onto which user interfaces and information of various types are projected. Interaction with the multitude of computing systems with which aspects of the disclosure may be practiced include, keystroke entry, touch screen entry, voice or other audio entry, gesture entry where an associated computing device is equipped with detection (e.g., camera) functionality for capturing and interpreting user gestures for controlling the functionality of the computing device, and the like.

Aspects of the present disclosure, for example, are described above with reference to block diagrams and/or operational illustrations of methods, systems, and computer program products according to aspects of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more aspects provided in this application are not intended to limit or restrict the scope of the disclosure as claimed in any way. The aspects, examples, and details provided in this application are considered sufficient to convey possession and enable others to make and use claimed aspects of the disclosure. The claimed disclosure should not be construed as being limited to any aspect, example, or detail provided in this application. Regardless of whether shown and described in combination or separately, the various features (both structural and methodological) are intended to be selectively included or omitted to produce an aspect with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

In addition, the aspects and functionalities described herein may operate over distributed systems (e.g., cloud-based computing systems), where application functionality, memory, data storage and retrieval and various processing functions may be operated remotely from each other over a distributed computing network, such as the Internet or an intranet. User interfaces and information of various types may be displayed via on-board computing device displays or via remote display units associated with one or more computing devices. For example, user interfaces and information of various types may be displayed and interacted with on a wall surface onto which user interfaces and information of various types are projected. Interaction with the multitude of computing systems with which aspects of the disclosure may be practiced include, keystroke entry, touch screen entry, voice or other audio entry, gesture entry where an associated computing device is equipped with detection (e.g., camera) functionality for capturing and interpreting user gestures for controlling the functionality of the computing device, and the like.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Any of the steps, functions, and operations discussed herein can be performed continuously and automatically.

The example systems and methods of this disclosure have been described in relation to computing devices. However, to avoid unnecessarily obscuring the present disclosure, the preceding description omits several known structures and devices. This omission is not to be construed as a limitation. Specific details are set forth to provide an understanding of the present disclosure. It should, however, be appreciated that the present disclosure may be practiced in a variety of ways beyond the specific detail set forth herein.

Furthermore, while the example aspects illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed configurations and aspects.

Several variations and modifications of the disclosure can be used. It would be possible to provide for some features of the disclosure without providing others.

In yet another configurations, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Example hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another configuration, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another configuration, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

The disclosure is not limited to standards and protocols if described. Other similar standards and protocols not mentioned herein are in existence and are included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

In accordance with at least one example of the present disclosure, a method for for predicting an odorant-receptor interaction is provided. The method includes receiving a first input indicating an odorant, determining molecular features of the odorant, receiving a second input indicating an olfactory receptor, determining protein features of the olfactory receptor, generating an odorant weight vector and an olfactory receptor weight vector based on the molecular features and the protein features, and determining a predicted interaction between the odorant and the olfactory receptor based on the odorant weight vector and the olfactory receptor weight vector.

In accordance with at least one aspect of the above method, the method may further include storing the predicted interaction between odorant and the olfactory receptor in a database.

In accordance with at least one aspect of the above method, the method may further include receiving an input from a user indicating a desired olfactory percept, determining one or more odorants that are predicted to be associated with the desired olfactory percept based at least in part on the database, and providing a list of one or more odorants to the user.

In accordance with at least one aspect of the above method, the method may further include where determining the molecular features of the odorant comprises converting a chemical structure of the odorant into a graph representation and generating the molecular features of the odorant using a graph neural network (GNN), wherein the molecular features are represented in a two-dimensional tensor.

In accordance with at least one aspect of the above method, the method may further include where determining the protein features of the olfactory receptor comprises determining the protein features of the olfactory receptor by passing a sequence of the olfactory receptor into a protein language model.

In accordance with at least one aspect of the above method, the method may further include where the odorant weight vector represents atoms of the odorant in an order of importance when interacting with the olfactory receptor.

In accordance with at least one aspect of the above method, the method may further include determining a weight for each atom of odorant by prioritizing atoms that are more likely to interact with the olfactory receptor based on the molecular features and the protein features, wherein the weight assigned to each atom is a probability that the corresponding atom is likely to interact with the olfactory receptor.

In accordance with at least one aspect of the above method, the method may further include where the olfactory receptor weight vector represents residues of the olfactory receptor in an order of importance when interacting with the odorant.

In accordance with at least one aspect of the above method, the method may further include determining a weight for each residue of olfactory receptor by prioritizing residues that are more likely to interact with the odorant based on the molecular features and the protein features, wherein the weight assigned to each residue is a probability that the corresponding residue is likely to interact with the odorant.

In accordance with at least one aspect of the above method, the method may further include where determining the predicted interaction between the odorant and the olfactory receptor based on the odorant weight vector and the olfactory receptor weight vector comprises concatenating the odorant weight vector and the olfactory receptor weight vector and predicting whether the odorant interacts with the olfactory receptor using a multi-layer neural network.

In accordance with at least one aspect of the above method, the method may further include where the predicted interaction is a probability that the odorant interacts with the olfactory receptor.

In accordance with at least one example of the present disclosure, a method for determining predicted olfactory perception is provided. The method includes receiving an input indicating an odorant, generating an odorant vector representing the odorant, generating an olfactory receptor vector indicating which olfactory receptors of a plurality of olfactory receptors are predicted to be activated by the odorant, and determining one or more predicted olfactory percepts associated with the odorant based on the odorant vector and the olfactory receptor vector.

In accordance with at least one aspect of the above method, the method may further include storing a mapping between odorant and one or more olfactory receptors predicted to be activated by the odorant and the one or more predicted olfactory percepts in a database.

In accordance with at least one aspect of the above method, the method may further include receiving an input from a user indicating a desired olfactory percept, determining one or more odorants that are predicted to be associated with the desired olfactory percept based at least in part on the mapping, and providing a list of one or more odorants to the user.

In accordance with at least one aspect of the above method, the method may further include where generating the odorant vector representing the odorant comprises converting a chemical structure of the odorant into a graph representation and generating the odorant vector using a graph neural network (GNN), wherein the odorant vector is a vector representation of the odorant.

In accordance with at least one aspect of the above method, the method may further include where generating the olfactory receptor vector comprises inputting sequences of preselected olfactory receptors into an interaction prediction model and generating the olfactory receptor vector using an interaction prediction model, wherein the olfactory receptor vector is a vector representation indicative of whether each of the plurality of olfactory receptors is likely to be activated by the odorant.

In accordance with at least one aspect of the above method, the method may further include where the interaction prediction model is trained to determine that an olfactory receptor of the plurality of olfactory receptors is likely to be activated by the odorant if a probability of interactions between the odorant and the corresponding olfactory receptor is above a predetermined threshold.

In accordance with at least one aspect of the above method, the method may further include where the interaction prediction model is trained to predict probabilities of interactions between the odorant and each of the plurality of olfactory receptors.

In accordance with at least one aspect of the above method, the method may further include where the interaction prediction model is trained to determine importance of atoms of the odorant and residues of each of the plurality of olfactory receptors when predicting a probability of whether the odorant interacts with the each of the plurality of olfactory receptors.

In accordance with at least one aspect of the above method, the method may further include where determining the one or more predicted olfactory percepts associated with the odorant comprises concatenating the odorant vector and the olfactory receptor vector and passing through a neural network to generate the one or more predicted olfactory percepts that are associated with the odorant.

The present disclosure, in various configurations and aspects, includes components, methods, processes, systems and/or apparatus substantially as depicted and described herein, including various combinations, subcombinations, and subsets thereof. Those of skill in the art will understand how to make and use the systems and methods disclosed herein after understanding the present disclosure. The present disclosure, in various configurations and aspects, includes providing devices and processes in the absence of items not depicted and/or described herein or in various configurations or aspects hereof, including in the absence of such items as may have been used in previous devices or processes, e.g., for improving performance, achieving ease, and/or reducing cost of implementation.

What is claimed is:

1. A method for predicting an odorant-receptor interaction, the method comprising:
receiving a first input indicating an odorant;
determining molecular features of the odorant;
receiving a second input indicating an olfactory receptor;
determining protein features of the olfactory receptor;
generating an odorant weight vector and an olfactory receptor weight vector based on the molecular features and the protein features; and
determining a predicted interaction between the odorant and the olfactory receptor based on the odorant weight vector and the olfactory receptor weight vector.

2. The method of claim 1, further comprising:
storing the predicted interaction between odorant and the olfactory receptor in a database.

3. The method of claim 2, further comprising:
receiving an input from a user indicating a desired olfactory percept;
determining one or more odorants that are predicted to be associated with the desired olfactory percept based at least in part on the database; and
providing a list of one or more odorants to the user.

4. The method of claim 1, wherein determining the molecular features of the odorant comprises converting a chemical structure of the odorant into a graph representation and generating the molecular features of the odorant using a graph neural network (GNN), wherein the molecular features are represented in a two-dimensional tensor.

5. The method of claim 1, wherein determining the protein features of the olfactory receptor comprises determining the protein features of the olfactory receptor by passing a sequence of the olfactory receptor into a protein language model.

6. The method of claim 1, wherein the odorant weight vector represents atoms of the odorant in an order of importance when interacting with the olfactory receptor.

7. The method of claim 6, further comprising:
determining a weight for each atom of odorant by prioritizing atoms that are more likely to interact with the olfactory receptor based on the molecular features and the protein features,
wherein the weight assigned to each atom is a probability that the corresponding atom is likely to interact with the olfactory receptor.

8. The method of claim 1, wherein the olfactory receptor weight vector represents residues of the olfactory receptor in an order of importance when interacting with the odorant.

9. The method of claim 8, further comprising:
determining a weight for each residue of olfactory receptor by prioritizing residues that are more likely to interact with the odorant based on the molecular features and the protein features,
wherein the weight assigned to each residue is a probability that the corresponding residue is likely to interact with the odorant.

10. The method of claim 1, wherein determining the predicted interaction between the odorant and the olfactory receptor based on the odorant weight vector and the olfactory receptor weight vector comprises concatenating the odorant weight vector and the olfactory receptor weight vector and predicting whether the odorant interacts with the olfactory receptor using a multi-layer neural network.

11. The method of claim 1, wherein the predicted interaction is a probability that the odorant interacts with the olfactory receptor.

12. A method for determining predicted olfactory perception, the method comprising:
receiving an input indicating an odorant;
generating an odorant vector representing the odorant;
generating an olfactory receptor vector indicating which olfactory receptors of a plurality of olfactory receptors are predicted to be activated by the odorant; and
determining one or more predicted olfactory percepts associated with the odorant based on the odorant vector and the olfactory receptor vector.

13. The method of claim 12, further comprising:
storing a mapping between odorant and one or more olfactory receptors predicted to be activated by the odorant and the one or more predicted olfactory percepts in a database.

14. The method of claim 13, further comprising:
receiving an input from a user indicating a desired olfactory percept;
determining one or more odorants that are predicted to be associated with the desired olfactory percept based at least in part on the mapping; and
providing a list of one or more odorants to the user.

15. The method of claim 12, wherein generating the odorant vector representing the odorant comprises converting a chemical structure of the odorant into a graph representation and generating the odorant vector using a graph neural network (GNN), wherein the odorant vector is a vector representation of the odorant.

16. The method of claim 12, wherein generating the olfactory receptor vector comprises inputting sequences of preselected olfactory receptors into an interaction prediction model and generating the olfactory receptor vector using an interaction prediction model, wherein the olfactory receptor vector is a vector representation indicative of whether each of the plurality of olfactory receptors is likely to be activated by the odorant.

17. The method of claim 16, wherein the interaction prediction model is trained to determine that an olfactory receptor of the plurality of olfactory receptors is likely to be activated by the odorant if a probability of interactions between the odorant and the corresponding olfactory receptor is above a predetermined threshold.

18. The method of claim 16, wherein the interaction prediction model is trained to predict probabilities of interactions between the odorant and each of the plurality of olfactory receptors.

19. The method of claim 18, wherein the interaction prediction model is trained to determine importance of atoms of the odorant and residues of each of the plurality of olfactory receptors when predicting a probability of whether the odorant interacts with the each of the plurality of olfactory receptors.

20. The method of claim 16, wherein determining the one or more predicted olfactory percepts associated with the odorant comprises concatenating the odorant vector and the olfactory receptor vector and passing through a neural network to generate the one or more predicted olfactory percepts that are associated with the odorant.
